(19) United States
(12) Patent Application Publication
(10) Pub. No.: US 2022/0202310 A1
(43) Pub. Date: Jun. 30, 2022

(54) Test station for screening of COVID-19 and other respiratory conditions

(71) Applicant: SANOLLA LTD., Nesher (IL)

(72) Inventor: Doron Adler, Haifa (IL)

(21) Appl. No.: 17/138,986

(22) Filed: Dec. 31, 2020

(51) Int. Cl. A61B 5/08; A61B 5/00; G01S 15/89

(52) U.S. Cl. CPC A61B 5/08; A61B 5/68; A61B 2562/0233; G01S 15/8909; A61B 5/746

(57) ABSTRACT

Apparatus for medical screening includes a plurality of acoustic transducers, which are configured to sense acoustic waves emitted from a body of a subject and to output signals in response thereto. A frame is configured to position the acoustic transducers in proximity to a thorax of the subject, so that the acoustic transducers receive the acoustic waves emitted from different, respective locations on the thorax. Processing circuitry is configured to collect and process the signals so as to assess a respiratory condition of the subject and to issue an alarm upon detecting a pathological respiratory condition, such as a suspicion of COVID-19.

### FIELD OF THE INVENTION

The present invention relates generally to methods and apparatus for medical diagnosis, and particularly to diagnosis of medical conditions based on acquisition and processing of acoustic signals.

### BACKGROUND

Auscultation has been a key technique in medical diagnosis for centuries. In auscultation, the medical practitioner listens to the internal sounds of the body, typically using a stethoscope. Auscultation is most commonly performed for the purpose of examining the circulatory and respiratory systems, and thus diagnosing conditions of the heart and lungs in particular. In more recent years, electronic stethoscopes and methods of digital processing of body sounds have become available, in order to enhance and supplement the practitioner's auditory capabilities.

For example, U.S. Patent Application Publication 2018/0228468, whose disclosure is incorporated herein by reference, describes an acoustic transducer, which is configured to sense infrasonic waves emitted from a body of a living subject. In some embodiments, processing circuitry computes and displays acoustic spectral patterns, referred to as “signatures,” based on the electrical signal output by the acoustic transducer. The inventors found that certain respiratory pathologies are characterized by distinct signatures.

PCT International Publication WO 2017/141165, whose disclosure is incorporated herein by reference, describes a digital stethoscope that allows the capture of both sonic and infrasonic sounds emanating from a human body, such as from the lungs of a human body. The stethoscope comprises a membrane, such as a circular membrane, which is typically fastened, at its edges, to a frame. As the membrane contacts the body, sound waves from the body—including both sonic and infrasonic sound waves—impinge on the membrane, causing deflections of the membrane. These deflections are then converted into digital signals that represent the original sound waves, including the infrasonic components of these sound waves. These signals may then be played to a user, analyzed by a processor (e.g., such as to automatically perform a diagnosis), and/or stored for later analysis.

PCT International publication WO 2019/048960, whose disclosure is incorporated herein by reference, describes medical devices that offer functionalities of electronic stethoscopes with enhanced diagnostic capabilities and ease of use. The devices include a case, whose front surface is brought into contact with the body of a living subject. The case contains a microphone, which senses acoustic waves emitted from the body, as well as a proximity sensor, which outputs a proximity signal indicative of contact between the front surface of the case and the body.

### SUMMARY

Embodiments of the present invention that are described hereinbelow provide methods and apparatus for acquisition and analysis of acoustic signals from the body.

There is therefore provided, in accordance with an embodiment of the invention, apparatus for medical screening, including a plurality of acoustic transducers, which are configured to sense acoustic waves emitted from a body of a subject and to output signals in response thereto. A frame is configured to position the acoustic transducers in proximity to a thorax of the subject, so that the acoustic transducers receive the acoustic waves emitted from different, respective locations on the thorax. Processing circuitry is configured to collect and process the signals so as to assess a respiratory condition of the subject and to issue an alarm upon detecting a pathological respiratory condition.

In a disclosed embodiment, the acoustic transducers are configured to sense infrasonic waves emitted from the body, and the processing circuitry is configured to assess the respiratory condition responsively to a feature of the infrasonic waves.

In some embodiments, the acoustic transducers are configured to contact the thorax of the subject while receiving the acoustic waves. Alternatively or additionally, the acoustic transducers are configured to sense the acoustic waves without contacting the thorax of the subject.

In a disclosed embodiment, the frame is configured as a booth, in which the subject stands so that the acoustic transducers are positioned in proximity to the thorax.

In some embodiments, the frame is configured to position the acoustic transducers so as to receive the acoustic waves from a dorsal surface of the thorax. In a disclosed embodiment, the frame comprises positioning arms, to which the acoustic transducers are attached and which are configured to move the acoustic transducers into contact with the dorsal surface at the respective locations. In one embodiment, the plurality of the acoustic transducers comprises four acoustic transducers, and the respective locations comprise first and second locations on upper left and upper right sides of the dorsal surface and third and fourth locations on lower left and lower right sides of the dorsal surface.

Additionally or alternatively, the apparatus includes an optical sensor, which is configured to sense a dimension of the body of the subject, wherein the processing circuitry is configured to drive the frame to position the acoustic transducers responsively to the sensed dimension.

In a disclosed embodiment, the processing circuitry is configured to issue the alarm when the signals are indicative of symptoms of a coronavirus.

There is also provided, in accordance with an embodiment of the invention, a method for medical screening, which includes positioning a plurality of acoustic transducers in proximity to different, respective locations on a thorax of a subject. Signals are output from the acoustic transducers at the respective locations in response to acoustic waves emitted from a body of the subject. The signals are processed so as to assess a respiratory condition of the subject and to issue an alarm upon detecting a pathological respiratory condition.

The present invention will be more fully understood from the following detailed description of the embodiments thereof, taken together with the drawings in which:

### BRIEF DESCRIPTION OF THE DRAWINGS

FIG. 1 is a schematic pictorial illustration of a system for medical screening, in accordance with an embodiment of the invention;

FIG. 2 is a schematic frontal view of a medical screening booth, in accordance with an embodiment of the invention;

FIGS. 3A and 3B are schematic front and rear views of a subject undergoing screening in the booth of FIG. 2;

FIGS. 4A and 4B are schematic top views of the booth of FIG. 2 in standby and active configurations, respectively;

FIG. 5A is a schematic frontal view of a medical screening booth, in accordance with another embodiment of the invention; and

FIG. 5B is a schematic top view of the booth of FIG. 5A during screening of a subject.

### DETAILED DESCRIPTION OF EMBODIMENTS

As a result of the worldwide COVID-19 epidemic, people who seek to enter public facilities or to pass from one country to another are subject to strict screening and clearance procedures. These procedures include, for example, testing body temperature and throat cultures (such as a PCR test). Temperature measurements are not sufficiently sensitive and specific, however, while throat cultures take a long time to process. There is thus a need for mass screening tools that can detect COVID-19 and other contagious respiratory diseases rapidly, with high sensitivity and specificity.

The inventors have found that sensing and analysis of acoustic waves emitted from the thorax of human subjects—and particularly of infrasonic waves, at frequencies below the range of human hearing—can be used to diagnose diseases such as COVID-19 rapidly and reliably. To carry out this procedure, one or more acoustic transducers acquire breath sounds from multiple locations on the thorax, for example four locations on the back of the subject (i.e., on the dorsal surface of the thorax). A processor digitizes and extracts features from the signals in the time domain, frequency domain, or both, including infrasonic features, and inputs these features to a software-based classifier, which is trained to distinguish COVID-19 on the basis of these features. Techniques that can be used in this sort of feature extraction and classification procedure are described, for example, in the above-mentioned US and PCT patent applications, as well as in U.S. Provisional Patent Application 63/119,677, filed Dec. 1, 2020, which is incorporated herein by reference.

In embodiments of the present invention that are described herein, the inventors have applied these techniques for acquisition and processing of acoustic signals to create a station that can be used for rapid, sensitive mass screening, for example in airports and at entries to other public facilities. In these embodiments, a frame positions multiple acoustic transducers in proximity to the thorax of the subject undergoing screening, so that the transducers receive acoustic waves emitted from different, respective locations on the thorax. The frame may advantageously be configured as a booth, in which the subject stands during screening. An optical sensor, such as a camera, senses the dimensions of the subject's body. Moving components of the frame, such as positioning arms, position the sensors at the appropriate locations in accordance with the sensed dimensions.

Processing circuitry collects and processes the signals output by the transducers so as to assess the subject's respiratory condition and to issue an alarm upon detecting a pathological condition. When multiple transducers, for example four transducers positioned against the subject's back, are used to acquire acoustic waves simultaneously, the entire test can be completed in about a minute or less.

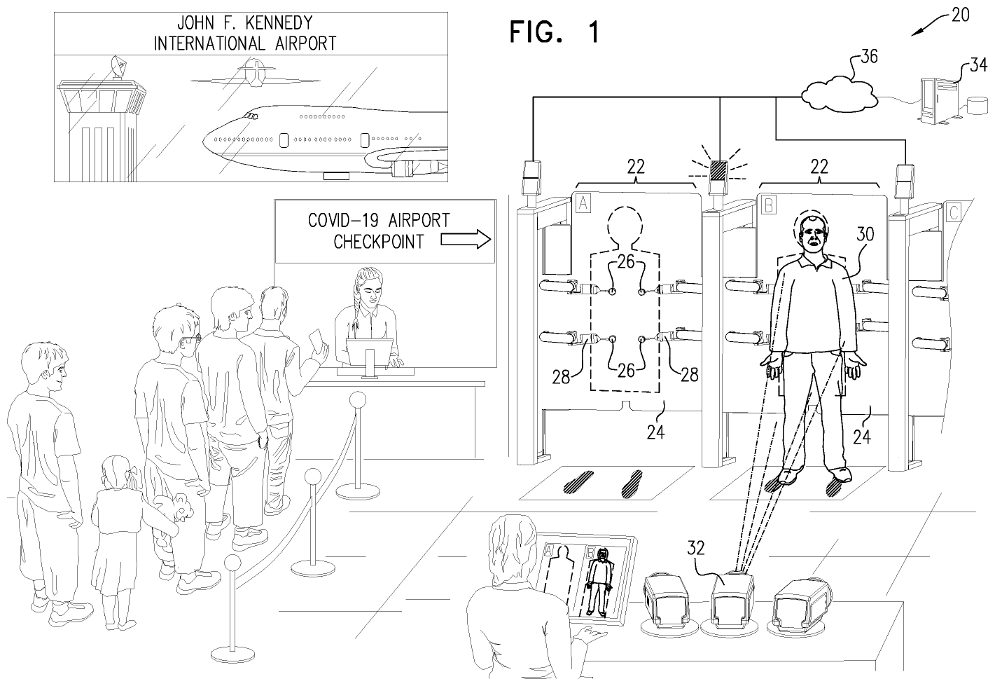

FIG. 1 is a schematic pictorial illustration of a system 20 for medical screening, in accordance with an embodiment of the invention. System 20 comprises one or more booths 22, having respective frames 24. Each booth 22 comprises multiple acoustic transducers 26, for example four transducers, which are attached to respective positioning arms 28.

When a screening subject 30 enters booth 22, an optical sensor, such as a camera 32, captures an image of the subject. Processing circuitry 34 processes the image in order to sense the dimensions, such as the height and width, of the subject's body. Alternatively or additionally, other sorts of sensors, such as photocells or proximity sensors, may be used to sense the body dimensions. Processor 34 then drives frame 24 and positioning arms 28 to position acoustic transducers 26 so as to receive acoustic waves from the subject's back. Transducers 26 may comprise, for example, vibrating membranes as described in the above-mentioned PCT International Publication WO 2017/141165. In this case, positioning arms 28 move transducers 26 into contact with the subject's back at the appropriate locations. (Such transducers are sufficiently sensitive to contact the back and receive the acoustic waves from the body through light clothing.) Alternatively or additionally, an operator of system 20 may control the positioning of the transducers.

Processing circuitry 34 receives the signals output by transducers 26 and processes the signals in order to assess the respiratory condition of subject 30. The processing circuitry extracts features from the signals, such as the time- and/or frequency domain features mentioned above, and applies a classifier to these features in order to classify the subject's respiratory condition as normal or pathological. Examples of such features are detailed in the above-mentioned provisional patent application and include values of average, median, standard deviation, and other statistical parameters in the time domain. In the frequency domain, the features may include dominant frequencies and their magnitudes, as well as Mel-frequency cepstral coefficients. Processing circuitry 34 may input these features to a classifier based on a suitably-trained neural network. Additionally or alternatively, processing circuitry 34 may compute and use features of the “signatures” of the acoustic signals, as described in the above-mentioned U.S. Patent Application Publication 2018/0228468. In any case, upon detecting a pathological condition, such as a pattern of features that is indicative of COVID-19 or another coronavirus, processing circuitry 34 issues an alarm.

In the pictured example, processing circuitry 34 comprises a general-purpose computer, which is connected to booths 22 via a network 36. The computer is programmed in software to carry out the control, signal processing, and communication functions described herein. The software may be downloaded to the computer in electronic form, over a network, for example, or it may alternatively or additionally be stored on tangible, non-transitory computer-readable media, such as optical, magnetic, or electronic storage media. Alternatively or additionally, a local processor associated with each of booths 22 may be used to perform at least some of the purposes of processing circuitry 34. Further alternatively or additionally, at least some of the functions of processing circuitry 34 may be carried out by a digital signal processor or by digital logic circuits, which may be hard-wired or programmable.

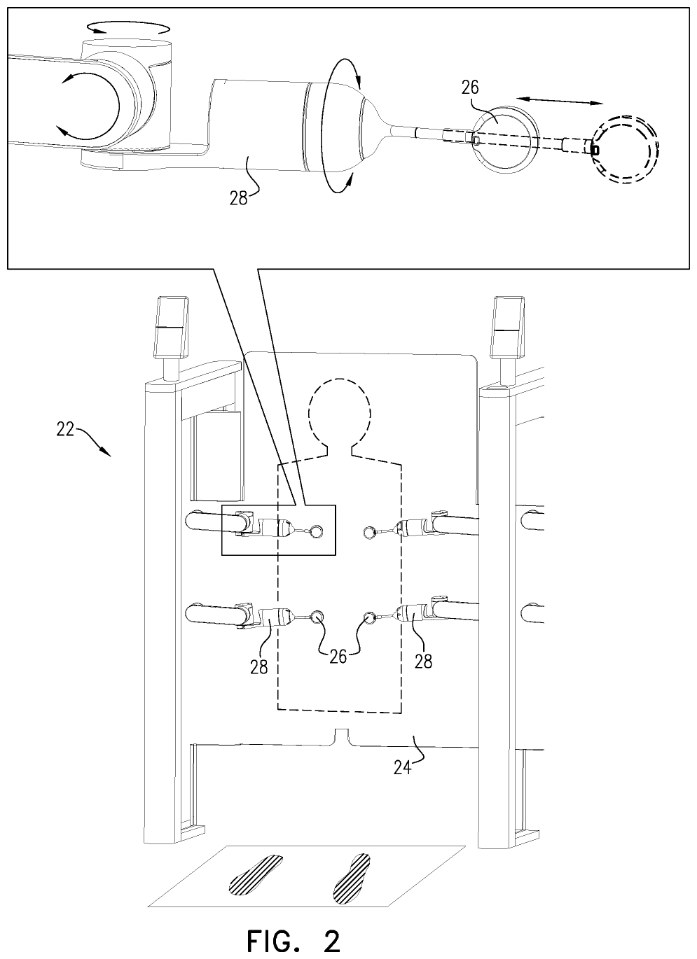

FIG. 2 is a schematic frontal view of screening booth 22, in accordance with an embodiment of the invention. An inset in this figure illustrates the operation of positioning arms 28. In this example, arms 28 are capable of rotation about three axes, as well as extension. Consequently, they can adjust the locations of transducers 26 in three dimensions in order to accommodate subjects having a wide range of different heights, widths, and body shapes.

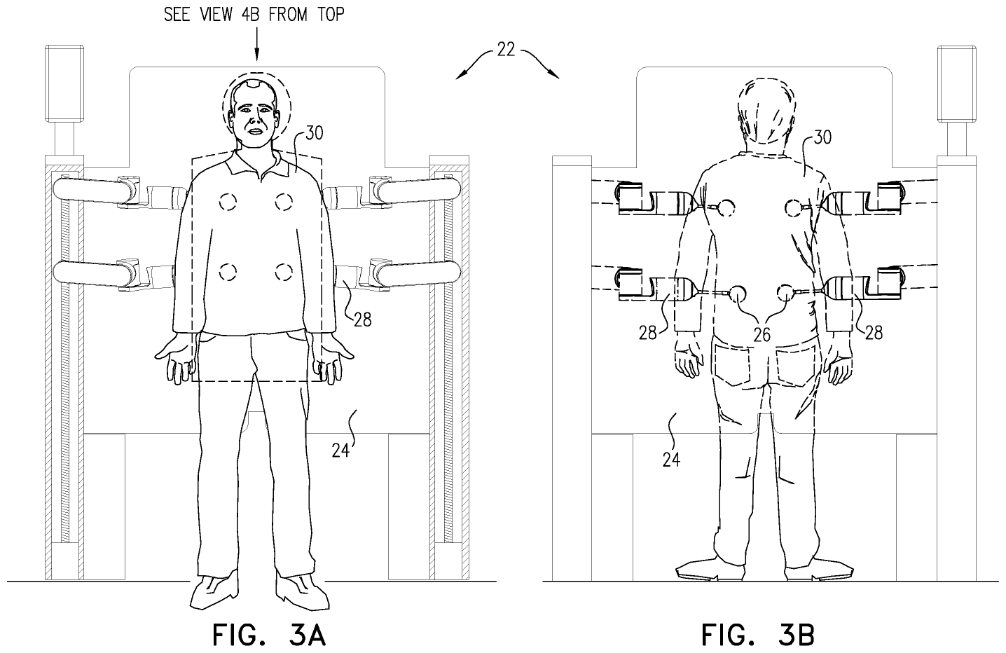

FIGS. 3A and 3B are schematic front and rear views, respectively, of subject 30 undergoing screening in booth 22, in accordance with an embodiment of the invention. The subject stands in the booth facing outward. Positioning arms 28 position transducers 26 to sense acoustic waves at four locations on the subject's back: first and second locations on upper left and upper right sides of the dorsal surface and third and fourth locations on lower left and lower right sides of the dorsal surface. The inventors have found this constellation of transducer locations to give good results in detecting and classifying respiratory pathologies. Alternatively, a larger or smaller number of transducers may be used, along with sensing at a larger or smaller number of locations. Further alternatively or additionally, the booth may be configured to apply one or more transducers to the frontal surface of the thorax.

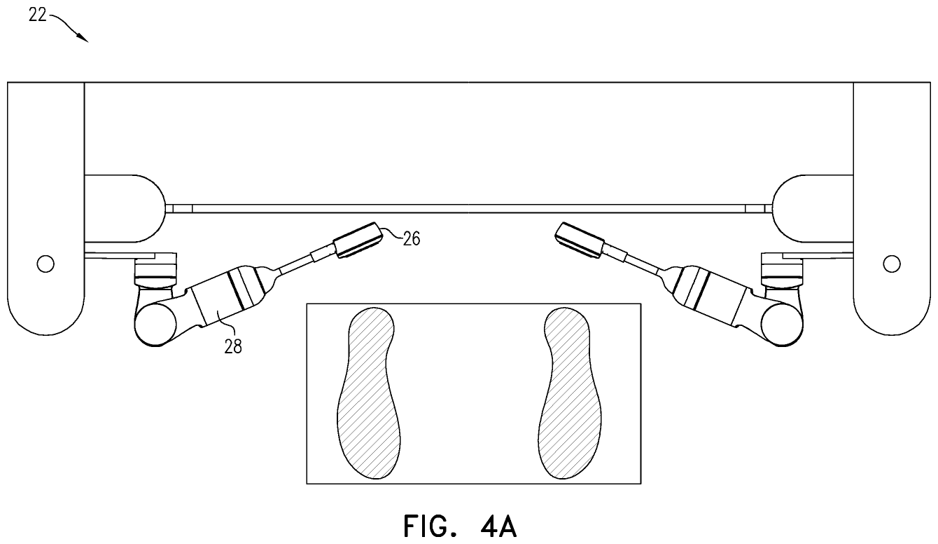

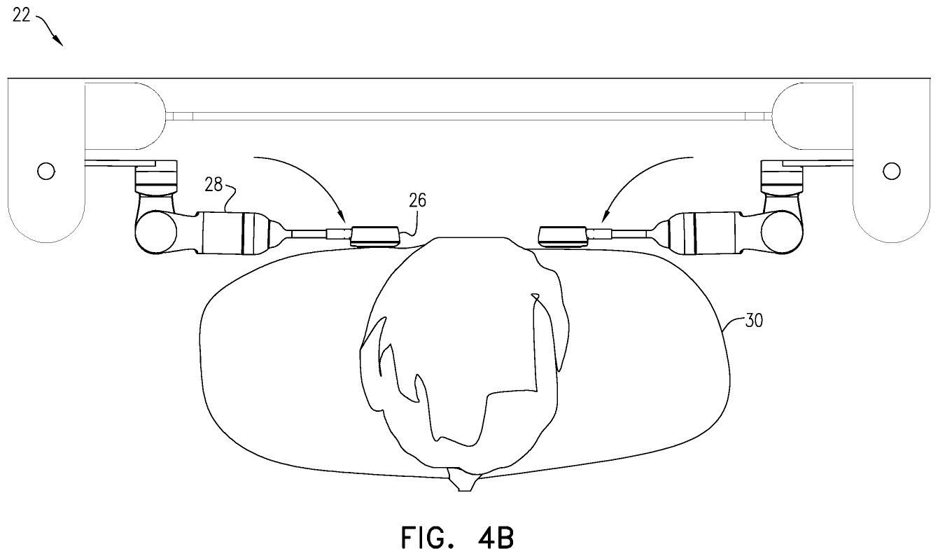

FIGS. 4A and 4B are schematic top views of booth 22 in standby and active configurations, respectively, in accordance with an embodiment of the invention. In the standby configuration of FIG. 4A, before a subject enters the booth, arms 28 retract transducers 26. Once subject 30 has positioned himself in booth 22, as shown in FIG. 4B, arms 28 adjust and rotate transducers 26 into place in contact with the subject's back. The processing circuitry may be assisted in positioning transducers 26 automatically in this manner both by images captured by camera 32 (FIG. 1) and by other sensors, such as proximity and pressure sensors (not shown) associated with transducers 26. These latter types of sensors are also useful in ensuring that transducers 26 are properly in contact with the subject's back in order to sense reliably the acoustic waves emitted from the body.

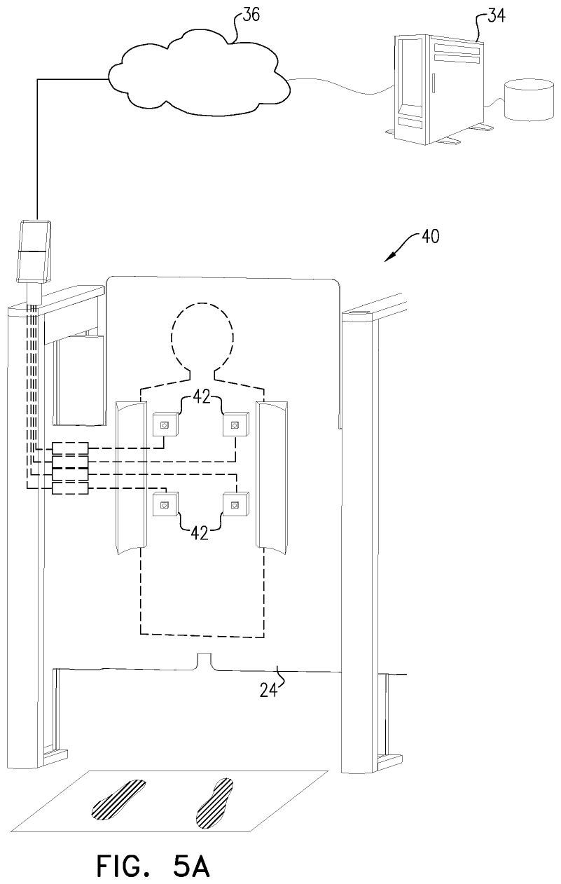

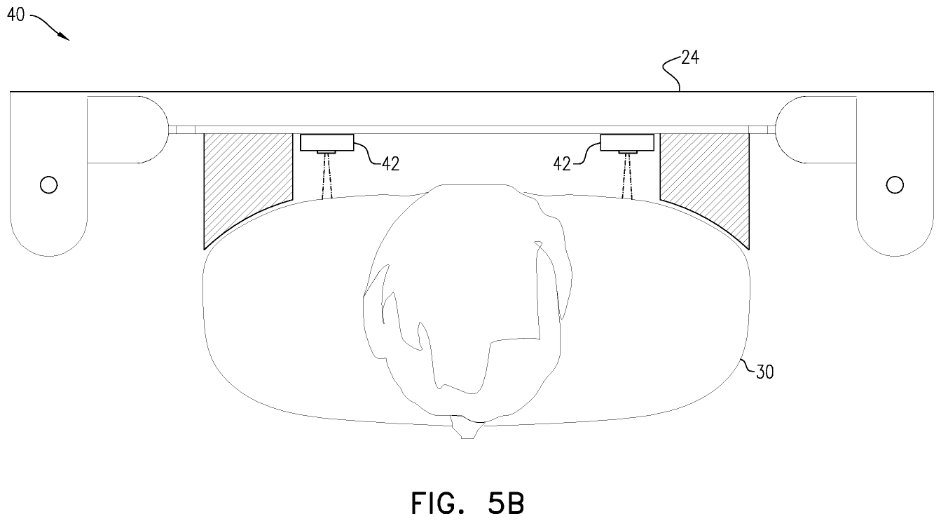

FIGS. 5A and 5B schematically illustrate a medical screening booth 40, in accordance with another embodiment of the invention. FIG. 5A is a frontal view of booth 40 in a standby mode, while FIG. 5B is a top view of booth 40 in an active mode, with subject 30 in the booth. Booth 40 may be used instead of booth 22 or in addition to booth 22 in system 20 (FIG. 1).

Booth 40 comprises non-contact acoustic transducers 42, which sense the acoustic waves emitted from the thorax of subject 30 without contacting the subject. For example, transducers 42 may comprise optical sensors, which transmit respective beams of radiation, such as infrared or millimeter-wave radiation, toward the surface of the subject's body and receive the radiation reflected back from the surface. In this case, each transducer 42 may comprise a small interferometer, which mixes the transmitted and received beams in order to sense small vibrations of the skin surface and extract the frequencies and amplitudes of the acoustic waves giving rise to these vibrations. Alternatively, transducers may comprise non-contact sensors of other kinds.

The use of non-contact sensors simplifies the mechanical design of booth 40 and may be beneficial for subjects who find the contact of the sensors with their backs objectionable. In an alternative embodiment (not shown in the figures), a screening booth may contain a combination of contact-based and non-contact sensors to facilitate more extensive and versatile data collection.

Although the figures and the description above refer, for the sake of concreteness and clarity, to certain specific system and sensor configurations, the principles of the present invention may be applied in creating and configuring other sorts of respiratory screening systems, as will be apparent to those skilled in the art after reading the present description. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Apparatus for medical screening, comprising:
a plurality of acoustic transducers, which are configured to sense acoustic waves emitted from a body of a subject and to output signals in response thereto;
a frame, which is configured to position the acoustic transducers in proximity to a thorax of the subject, so that the acoustic transducers receive the acoustic waves emitted from different, respective locations on the thorax; and
processing circuitry, which is configured to collect and process the signals so as to assess a respiratory condition of the subject and to issue an alarm upon detecting a pathological respiratory condition.

2. The apparatus according to claim 1, wherein the acoustic transducers are configured to sense infrasonic waves emitted from the body, and the processing circuitry is configured to assess the respiratory condition responsively to a feature of the infrasonic waves.

3. The apparatus according to claim 1, wherein the acoustic transducers are configured to contact the thorax of the subject while receiving the acoustic waves.

4. The apparatus according to claim 1, wherein the acoustic transducers are configured to sense the acoustic waves without contacting the thorax of the subject.

5. The apparatus according to claim 1, wherein the frame is configured as a booth, in which the subject stands so that the acoustic transducers are positioned in proximity to the thorax.

6. The apparatus according to claim 1, wherein the frame is configured to position the acoustic transducers so as to receive the acoustic waves from a dorsal surface of the thorax.

7. The apparatus according to claim 6, wherein the frame comprises positioning arms, to which the acoustic transducers are attached and which are configured to move the acoustic transducers into contact with the dorsal surface at the respective locations.

8. The apparatus according to claim 7, wherein the plurality of the acoustic transducers comprises four acoustic transducers, and wherein the respective locations comprise first and second locations on upper left and upper right sides of the dorsal surface and third and fourth locations on lower left and lower right sides of the dorsal surface.

9. The apparatus according to claim 1, and comprising an optical sensor, which is configured to sense a dimension of the body of the subject, wherein the processing circuitry is configured to drive the frame to position the acoustic transducers responsively to the sensed dimension.

10. The apparatus according to claim 1, wherein the processing circuitry is configured to issue the alarm when the signals are indicative of symptoms of a coronavirus.

11. A method for medical screening, comprising:
positioning a plurality of acoustic transducers in proximity to different, respective locations on a thorax of a subject;
outputting signals from the acoustic transducers at the respective locations in response to acoustic waves emitted from a body of the subject; and
processing the signals so as to assess a respiratory condition of the subject and to issue an alarm upon detecting a pathological respiratory condition.

12. The method according to claim 11, wherein outputting the signals comprises sensing infrasonic waves emitted from the body, and processing the signals comprises assessing the respiratory condition responsively to a feature of the infrasonic waves.

13. The method according to claim 11, wherein positioning the plurality of the acoustic transducers comprises bringing the acoustic transducers into contact with the thorax of the subject while receiving the acoustic waves.

14. The method according to claim 11, wherein positioning the plurality of the acoustic transducers comprises sensing the acoustic waves without contacting the thorax of the subject.

15. The method according to claim 11, wherein positioning the plurality of the acoustic transducers comprises mounting the acoustic transducers in a booth, in which the subject stands so that the acoustic transducers are positioned in proximity to the thorax.

16. The method according to claim 11, wherein positioning the plurality of the acoustic transducers comprises positioning the acoustic transducers so as to receive the acoustic waves from a dorsal surface of the thorax.

17. The method according to claim 16, wherein positioning the acoustic transducers comprises moving the acoustic transducers automatically into contact with the dorsal surface at the respective locations.

18. The method according to claim 17, wherein the plurality of the acoustic transducers comprises four acoustic transducers, and wherein the respective locations comprise first and second locations on upper left and upper right sides of the dorsal surface and third and fourth locations on lower left and lower right sides of the dorsal surface.

19. The method according to claim 11, wherein positioning the plurality of the acoustic transducers comprises sensing a dimension of the body of the subject, and positioning the acoustic transducers responsively to the sensed dimension.

20. The method according to claim 11, wherein processing the signals comprises issuing the alarm when the signals are indicative of symptoms of a coronavirus.
